(19) United States
(12) Patent Application Publication
(10) Pub. No.: US 2016/0256584 A1
(43) Pub. Date: Sep. 8, 2016

(54) COMPOSITIONS AND METHODS FOR THE ERADICATION OF ODORS

(71) Applicant: NBIP, LLC, Richardson, TX (US)

(72) Inventors: John W. Veenstra, Richardson, TX (US); Benny McKee, Arlington, TX (US)

(21) Appl. No.: 15/061,894

(22) Filed: Mar. 4, 2016

Related U.S. Application Data
(60) Provisional application No. 62/128,310, filed on Mar. 4, 2015.

(51) Int. Cl. A61L 9/014; A01N 25/08; A01N 37/36

(52) U.S. Cl. CPC A61L 9/014; A01N 37/36; A01N 25/08

(57) ABSTRACT

The disclosed compositions and methods aid in controlling or eliminating many common odors and bacteria and enzymes that cause odor. Odors are controlled or eliminated by bringing the compositions into contact or proximity with the odors. The compositions are stored in an absorbent carrier which is then placed in a position where it will come into contact with the odor or the source of the odor. The compositions stored in the absorbent material can be fully hydrated for use or hydrated and then dehydrated for use. The disclosed compositions comprise a biocidal system comprising one or more primary biocides, one or more pH buffers, and one or more surfactants. The composition may be placed in an aqueous solution.

### CROSS-REFERENCES TO RELATED APPLICATIONS

This Application claims the benefit under 35 U.S.C. §119(e) of U.S. Provisional Patent Application Ser. No. 62/128,310, filed Mar. 4, 2015, which is incorporated herein by reference in its entirety as if fully set forth herein.

### FIELD OF THE INVENTION

The present disclosure relates to the use of absorbent carriers to deliver compositions that are effective in controlling odor and the bacteria and enzymes that cause odor.

### BACKGROUND OF THE INVENTION

Odors can come from various sources. For example, odors may arise from food preparation, smoke, urine, feces, breath, fur, feathers, cages, pens, litter, etc. Effective odor control is an important objective in many consumer product applications designed for personal, household or institutional use, for example, fabric softening compositions, freshening compositions for fabrics and clothes, deodorant compositions for personal or household use, e.g., for deodorizing pet, tobacco, cooking and toilet smells, personal cleansing compositions and cosmetics for controlling body odor, dentifrice and mouthwash compositions for controlling mouth odor, laundry compositions designed to clean and freshen fabrics, manual and machine dishwashing compositions for cleaning and deodorizing dishes, plastic containers, etc., hard surface cleaning compositions for cleaning and deodorizing floors, carpets and other floor coverings, bleaching compositions for fabric or household use. Odor control is also very important in many industrial situations, for example in rendering plants, spent grain dryers, pharmaceutical plants and cellulose pumping.

In general terms, odor can be described as the subjective perception of the sense of smell. The original classification of odors recognized that many odors are repulsive or nauseous in nature and, by common agreement, would be described as ‘malodors’. Body fluids, for example, contain a variety of substances which are malodorous per se or which can over time form malodorous compounds by a fermentation or degradation mechanism, for example, volatile fatty acids (e.g. isovaleric acid), ammonia, amines (e.g. triethylamine), sulphur-containing compounds (e.g. mercaptans, sulphides), alcohols, ketones and aldehydes (e.g. furaldehyde). Apart from body fluids, there are many other potential environmental sources of malodors, e.g. tobacco and smoking, food and cooking smells, pets, animal rendering, etc. In general, the object of odor control is to provide a process or composition that will give a more acceptable perception of smell, whether as a result of dilution, removal of the offending substance, or counteraction or masking, or indeed by preventing the generation of malodors at source.

One approach that has been taken to the control of gaseous odors involves absorption or adsorption of the odor on high surface area porous substrates such as activated carbon and zeolites. The known sorbents (which term includes both absorbents and adsorbents), however, have only a limited efficacy for odor-control and in particular are relatively poor absorbers of higher molecular weight and/or hydrophobic malodor components. In other words, the known sorbents have a relatively limited odor-absorption spectrum.

Accordingly, the present invention provides odor-control compositions and a method of delivery. The present invention also provides a range of consumer product applications which utilize the odor-control agents. Thus, it would be desirable to have an effective delivery method for a composition that is capable of eradicating odors and/or the causes of odors before the odor begins to occur.

### SUMMARY OF THE INVENTION

In its broadest aspect, the present invention relates to the use of absorbent carriers that are used in conjunction with an odor eliminating composition that has been absorbed into the absorbent carrier, for the control and/or elimination odors. The disclosed compositions and methods control or eliminate many common odors and bacteria and enzymes that cause odor. The compositions are brought into contact with the odors by way of being stored in an absorbent carrier which is then placed in a position where it will come into contact with the odor or the source of the odor. The compositions stored in the absorbent material can be fully hydrated for use or hydrated and then dehydrated for use.

In a typical embodiment, the disclosed compositions include a biocidal system that includes one or more primary biocides, one or more pH buffers, and one or more surfactants, which are all in an aqueous composition.

In one embodiment, a method of controlling odors of an animate or inanimate object includes treating the object with a composition including large pore cross-linked polymer crystals.

In one embodiment, an odor-control composition including an odor-control agent is provided. Such compositions can take various forms including particulate solids, liquids, gels and aerosols.

The present invention also provides a variety of consumer and retail products designed for personal, household, industrial and institutional use, for example, freshening compositions for fabrics and clothes and odor-control and deodorant compositions for personal or household use. For example, compositions may be used for deodorizing pet, tobacco, cooking, and toilet smells.

The present disclosure addresses solutions to several unmet needs, such as, for example:

In some embodiments, an absorbent carrier is provided that can be hydrated with an odor eliminating composition for storage and later use to eradicate odor and the bacteria and enzymes that cause odor.

In some embodiments, compositions effective in killing one or more bacterial pathogens are provided. Non-limiting examples include: enterococci, group B. streptococci, E faecalis, coagulase negative staphylococci, Escherichia coli, (E. coli) and Klebsiella pneumoniae.

In some embodiments, compositions effective against environmental bacterial pathogens are provided. Non-limiting examples include: Streptococcus spp, Escherichia coli, Klebsiella species, A. pyogenes and Pseudomonas species.

In some embodiments, compositions effective against bacterial caused odors, such as, for example, ammonia, hydrogen sulfide, methyl mercaptan, allylmethyl sulfide and dimethyl sulfide are provided.

In some embodiments, compositions effective in preventing the spread of one or more odor-causing bacterial and/or enzymatic pathogens from an infected animal or human to other animals or humans or an apparatus that contacts any infected animals or humans are provided.

In some embodiments, compositions are provided for reducing ammonia from animal or human urine and excrement.

In some embodiments, compositions are provided for reducing and eliminating ammonia odor, food preparation odor, cooking odor and smoke odor.

In some embodiments, compositions are provided for reducing and eliminating other animal odors such as breath, fur and feathers.

### DETAILED DESCRIPTION

In this specification and in the claims that follow, reference will be made to a number of terms that shall be defined to have the following meanings:

The term “animal” as used herein means all pets, domesticated and non-domesticated animals, humans and all living beings that produce body odors or body fluids or excrement.

The term “excrement” as used herein means the urine or feces emanating from an animal.

Throughout this specification, unless the context requires otherwise, the word “comprise,” or variations such as “comprises,” “comprising,” “include,” “includes,” and “including” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “an aqueous agent” includes mixtures of two or more such carriers, and the like.

The term “effective amount” as used herein means “an amount of a composition as disclosed herein, effective at dosages and for periods of time necessary to achieve the desired results.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations by use of the antecedent “about,” it will be understood that the particular value forms another aspect. It will be further understood that the endpoints of each of the ranges are significant both in relation to the other endpoint and independently of the other endpoint.

A “weight percent” of a component, unless specifically stated to the contrary, is based on the total weight of the formulation or composition in which the component is included.

“Contacting” as used herein refers to applying the absorbent carrier containing the composition to the cause of the odor. By way of example, an animal or an animal's excrement, urine in synthetic turf, cat litter, carpet, pet bedding, etc. further examples, human hair removal, breath, colonics, urine and excrement, etc.

“Sufficient amount” and “sufficient time” as used herein refers to an amount and time needed to achieve the desired result or results, such as control, eradication and/or prevention of odors.

“Eradicate” and “eradication” and “eradicating” as used herein refers to elimination of odor and the bacteria and enzymes that cause odor without the use of harsh chemicals, synthetic smells, masking smells, cover-up smells or enzymes.

“Admixture” or “blend” as generally used herein means a physical combination of two or more different components.

“Controlled release” as used herein means the use of an absorbent material to regulate the release of an odor eradicating composition.

“Excipient” is used herein to include any other compound that may be contained in or on the microparticle, which is not a therapeutically or biologically active compound. As such, an excipient should be pharmaceutically or biologically acceptable or relevant (for example, an excipient should generally be non-toxic to the subject). “Excipient” includes a single such compound and is intended to include a plurality of excipients.

“Odor” is used herein to refer to any pungent smell or stench including bacterial and ammonia odors, associated with animals and their excrement, and other odors, such as environmental odors, including food preparations, cooking and smoke.

“Primary biocide” is used herein to refer to compounds that are biologically active against a primary pathogen.

“Primary pathogen” is used herein to refer to bacteria, enzymes, viruses, fungi or other biologically active microorganisms.

“Infused” as used herein means the process by which an absorbent carrier is hydrated with an odor eliminating composition. Hydration methods may include spraying, dipping, immersing, soaking, spray drying, humidifying, etc.

### Absorbent Carriers for Hydration by Treatment Compositions

An embodiment of the invention is directed to absorbent carriers that can be hydrated with a composition that can eradicate odors and the bacteria and enzymes that cause odors. In some embodiments, the absorbent carrier includes: organic materials, such as, for example, cotton, paper, charcoal, clay, soil, manure, walnut shells, wheat and wheat by products, and saw dust; inorganic materials such as cellulosic, polymers, hydrogels, upsalite, silica; and natural carriers, such as, for example, carbohydrates (including but not limited to disaccharides, maltodextrins, fibers, glucans, gums, starches, syrups and cellulose derivatives), lipids (including but not limited to polyethylene glycols, phospholipids, and fatty acids), proteins (including but not limited to gelatin, casein, and whey). In some embodiments, synthetic absorbent carriers include, but are not limited to, polymers, acrylates, polyvinyl alcohol, silicas/silicates, calcium chloride, and rock salt.

In some embodiments, the amount of the odor control composition in the absorbent carrier that is effective for controlling odors, includes the odor control composition in the absorbent carrier preferably in an amount of at least about 0.001%, more preferably from about 0.001% to about 90%, and most preferably from about 0.05% to about 50% by weight of composition.

In some embodiments, the absorbent carriers include large pore cross-linked polymer crystals. In some embodiments, the absorbent carriers include a pore diameter greater than about 10 Angstroms. In other embodiments, the absorbent carriers may include pore diameters of at least 13 Angstroms, at least 15 Angstroms, or at least 20 Angstroms. In an embodiment of the invention, the large pore cross-linked polymer crystals are polyacrylamide crystals.

In an embodiment of the invention, the polymer crystal in its dehydrated form is rehydrated with an odor eliminating liquid solution until saturated. The rehydrated polymer is subsequently dehydrated to form crystals containing a highly concentrated amount of the odor eliminating composition. The crystals can then be used in various odor-control situations. In some embodiments, the odor eliminating composition is a liquid with odor absorption and/or elimination properties that is used to rehydrate the polymer crystals. Illustrative compositions are described in more detail below.

In some embodiments, the polymer crystals are incorporated with a binder or a porous matrix material. In some embodiments, the polymer crystals are used in conjunction with another odor-control material. Suitable binders include inorganic materials such as sodium sulphate, clays, silica and/or metal oxides, such as alumina, titania, and/or zirconia. Other odor control materials include, for example, naturally occurring materials and/or gelatinous precipitates or gels. For example, gelatinous materials may include mixtures of silica and metal oxides. Naturally occurring materials may include, for example, naturally occurring clays such as bentonite and kaolin. Suitable organic binders may include cellulose derivatives such as carboxymethyl cellulose and water-soluble polymers such as sodium polyacrylate. Suitable porous matrices may include silica-alumina, silica-magnesia, silica zirconia, silica-thoria, silica-beryllia, silica-titania, as well as ternary compositions such as silica-alumina-thoria, silica-alumina-zirconia, silica-alumina-magnesia, and silica-magnesia-zirconia. Other odor-control agents suitable for use herein include small-pore zeolites such as Zeolite A, Zeolite X and Zeolite Y, activated carbon, charcoal and antimicrobial agents.

In some embodiments, a method of infusing or hydrating an absorbent carrier with an odor eradicating composition includes applying the composition onto the absorbent carrier. For example, the composition may be applied via a hose, nozzle, mister, fogger, hydrator, humidifier, immersing the absorbent carrier in the composition, spray-drying the composition onto the absorbent carrier, and the like. In a typical embodiment, spray-drying the composition includes applying the composition to the absorbent carrier and then allowing the absorbent carrier to dry, creating an absorbent carrier with odor eradicating properties.

In some embodiments, a method of dehydrating a previously hydrated absorbent carrier includes drying the hydrated absorbent carrier with, for example, heat, freeze drying, or air drying, or flash freezing.

### Treatment Compositions

Embodiments of the invention are directed to compositions for reducing odors associated with, for example, excrement, breath, fur, and feathers, where ammonia and other mal-odor build up is observed in humans and animals. The compositions may include from about 0.01% to about 20.0% by weight of a biocidal system. In a typical embodiment, the biocidal system may include from about 0.01% to about 25% by weight of a primary biocide, and at least about 0.01% to about 25% by weight of a pH buffer, where the pH buffer is a biocidal, dermal, non-corrosive acid composition having a proton count range of from 1.0×10²⁰ to 9.9×10²⁶, an embodied conductivity range of from 250 mV to 1500mV, and has a pH of less than 2.0 when the pH buffer is present at a concentration of 1% by weight. The composition may further include from about 0.01% to 2% by weight of a surfactant. The balance of the composition may include an aqueous agent. Other embodiments and combinations are possible as further disclosed herein.

Odor-control and deodorant compositions of the present invention generally include the odor-control agent in combination with a carrier therefor and/or a perfume. In one aspect of the invention, such compositions are utilized to reduce malodor on inanimate surfaces such as carpets, upholstery, fabrics, clothes, etc., for example, clothes that have been stored for a long period of time, or carpets that have been contaminated with environmental odors such as food odors, tobacco odors, etc. Such compositions can thus be used to restore and/or maintain freshness by reducing malodor without the need for cleaning, washing or dry cleaning.

Preferred compositions take the form of a fluid or sprayable aqueous composition which in use is dispensed onto fabrics, clothes, carpets, shoe inners, etc. to restore their freshness by reducing malodor impression. The compositions are designed to extend the wear and freshness of the treated articles between cleaning, washing or dry cleaning. In the case of compositions which additionally contain a perfume, articles treated therewith will stay fresher longer, and receive extra freshening effect via perfume release.

### Biocidal System

The disclosed compositions may include a biocidal system. In a typical embodiment, the biocidal system includes a primary biocide and a pH buffer.

### Primary Biocide

A first group of suitable biocides include quaternary ammonium compounds chosen from (C₁₂-C₁₄ alkyl)(C₁-C₂ dialkyl) benzyl ammonium salts, N-(C₁₂-C₁₈ alkyl) heteroaryl ammonium salts, and N-[(C₁₂-C₁₄ alkyl)(C₁-C₂ dialkyl)] heteroarylalkylene ammonium salts. Non-limiting examples of the (C₁₂-C₁₄ alkyl)(C₁-C₂ dialkyl) benzyl ammonium salts include (C₁₂-C₁₄ alkyl)dimethyl-benzyl ammonium chloride, (C₁₂-C₁₄ alkyl) dimethylbenzyl ammonium bromide, and (C₁₂-C₁₄ alkyl) dimethylbenzyl ammonium hydrogen sulfate. Non-limiting examples of the N-(C₁₂-C₁₈ alkyl)heteroaryl ammonium salts include cetyl pyridinium chloride, cetyl pyridinium bromide, and cetyl pyridinium hydrogen sulfide. For the N-(C₁₂-C₁₈ alkyl)heteroaryl ammonium salts other anions can be used.

Further examples of quaternary ammonium compounds suitable for use as the primary biocides include cetyltrimethylammonium chloride, stearyltrimethylammonium chloride, isostearyltrimethylammonium chloride, lauryltrimethylammonium chloride, behenyltrimethyl-ammonium chloride, octadecyltrimethylammonium chloride, cocoyltriinethylammonium chloride, cetyltrimethylammonium bromide, stearyltrimethylammonium bromide, lauryl-trimethylammonium bromide, isostearyllauryldimethylammonium chloride, dicetyldimethyl-ammonium chloride, distearyldimethylammonium chloride, dicocoyldimethylammonium chloride, gluconamidopropyldimethylhydroxyethylammonium chloride, di[polyoxyethylene(2)] oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryl-dimethyl(ethylbenzyl)ammonium chloride, behenamidopropyl-N,N-dimethyl-N-(2,3-dihydroxypropyl)ammonium chloride, tallowdimethylammoniopropyltrimethylammonium dichloride, and benzalconium chloride.

A second group of suitable biocides includes copper, zinc, and silver, salts of chlorides, chlorites, perchlorates, hypochlorites, sulfates, sulfites, bisulfates, bisulfites, nitrates, nitrites, and hydroxides. Also, colloid metal such as silver, gold, copper and zinc have superior biocidal properties. Colloidal silver, gold, copper, and zinc are extracted and created as ultrafine (0.010-0.001 micron) particles.

A third group of suitable biocides includes organic acids which are safe under the FDA GRAS guidelines for food production yet still effective in controlling bacteria.

The first group of suitable organic acids is Lactic, Acetic, Formic, Fumaric, Citric, Oxalic, Adipic, and Uric.

The second group of suitable organic acids is the carboxylic acids, whose acidity is associated with their carboxyl group —COOH. Sulfonic acids, containing the group —SO₂OH, are relatively stronger acids. The relative stability of the conjugate base of the acid determines its acidity. In some biological systems more complex organic acids such as L-lactic, citric, and D-glucuronic acids are formed. These use the hydroxyl or carboxyl group.

The third group of suitable organic acids are Humic, Sebacic, Stearic, Gallic, Palmitic, Caffeic, Glyoxylic, Fulvic, Carnosic, Anthranilic, Ellagic, Lipoic, Chlorogenic, Rosmarinic, Phosphoric, Methacrylic, Oleanic, Nitrohumic, Florocinnamic, Hexaflorosilicic, Hydrofluoric, Hydroxycitric and Silicofluoric.

The fourth group of suitable organic acids is fruit acids. The acids in fruits are chiefly acetic, malic, citric, tartaric, oxalic, mandelic, and in some instances boric. Malic acid is present in apples, pears, currants, blackberries, raspberries, quince, pineapple, cherries, and rhubarb. Citric acid is found in lemons, oranges, grapefruit, lemons, limes, quince, gooseberry, strawberry, raspberry, currant, and cranberry. Tartaric acid occurs in grapes. Boric acid is found in many fresh fruits and vegetables. Mandelic acid is present in almonds.

The fifth group of suitable organic acids is beta hydroxy acids, which is a type of phenolic acid. For example, salicylic acid is a colorless crystalline organic acid whose main active ingredient obtained from this source is a monohydroxiybenzoic acid.

The sixth group of suitable organic acids is a class of products that break biofilm. Biofilms are the protective layer/barrier that surround bacteria. Some species are not able to attach to a surface on their own but are often able to anchor themselves to the matrix or the bacteria cells. It is during this colonization that the cells are able to communicate via its quorum sensing ability. Once colonization has begun, the biofilm grows through a combination of cell division and recruitment. The final stage of biofilm formation is known as development and is the stage in which the biofilm is established and may only change in shape and size. The development of a biofilm may allow an aggregate cell colony to be increasingly resistant. A biofilm's hard protective surface can be broken by, for example, Lactobacillus sc Nisin which is produced by fermentation using the bacterium Lactococcus lactis. In various embodiments, the primary biocide may be chosen from several natural and/or organic acids that break the biofilm such as acids from fermented, such as, for example, Lactococcus lactis, Lactococcus cremoris, Lactococcus hordniae, Lactococcus garvieae, Lactococcus platarum, Lactococcus raffinolactis, or Lactococcus piscium products. Lactococcus lactis may be obtained from the culturing of Lactococcus lactis on natural substrates, such as milk or dextrose, and is not chemically synthesized. This is a peptide which is produced by the food grade dairy starter bacterium Lactococcus lactis.

A seventh group of suitable organic acids is natural enzymes. Enzymes are proteins that catalyze chemical reactions and range from just 62 amino acid residues. Typically, these are protease, lipase, diastase, and cellulase enzymes. Enzymes are usually very specific as to which reactions they catalyze and the substrates that are involved in these reactions. The shape, charge, and hydrophilic/hydrophobic nature characterize the enzymes. In some embodiments, the primary biocide is chosen from a wide range of natural enzymes such as proteolytic, amylolytic, cellulase, papin, invertase, lipolytic, pepsin, bromelain and lactase.

### pH Buffer

In a typical embodiment, the pH buffer is a biocidal, dermal, non-corrosive acid composition, having a proton count range of from 1.0×10²⁰ to 9.9×10²⁶, an embodied conductivity range of from 250 mV to 1500 mV and a 1% solution of the composition having a pH of under 2.0.

### Surfactant

A surfactant component includes ingredients that modify the water in the system making it suitable from use with several types of water such as hard water, soft water, sulfite contaminated water, rain water, pond water, well water or calcium rich water. This allows the embodiment composition to cling or stick to the animal's hair or skin, or excrement. Cationic surfactants of the quaternary ammonium compound referred to as “poly quats” are used in many hair shampoos and conditioners. These agents improve the body of the hair and reduce its static charge. Surfactant products often used for human hair treatment provide the cling or stickiness necessary for the application of the biocidal compositions to animals. Quaternary ammonium compounds which are more compatible with anionic surfactants generally have an inadequate conditioning effect.

In-order to improve the ability for an aqueous mixture to adhere to an animal, or its excrement, dialkyl diallyl ammonium chloride/acrylic acid-type polymers is added. This method for improving the stickiness properties of the composition encompasses adding an effective amount of a polymer that includes, for example, about 60% to about 99% (based on total polymer weight) of a quaternary diallyl dialkyl ammonium monomer, where alkyl groups are independently selected from alkyl groups of 1 to 18 carbon atoms, preferably C₁₄ alkyl, and where said quaternary diallyl dialkyl ammonium monomer's counterion may include conjugate bases of acids having an ionization constant greater than 10⁻¹³. In some embodiments, the conjugate bases of acids include fluoride, chloride, bromide, hydroxide, nitrate, acetate, hydrogen sulfate, and primary phosphates. The composition may further include about 1% to about 40% (based on total polymer weight) of an anionic monomer. In some embodiments, the anionic monomer may include acrylic acid and methacrylic acid; where the average molecular weight of said polymer ranges from about 50,000 to about 10,000,000, as determined by gel permeation chromatography.

The polymer base can also be a combination of one or more bases, for example, glycerol in combination with ethoxylated partial glyceride fatty acid esters. These include branched chain esters, ethoxylated partial glyceride fatty acid esters, protein derivatives, lanolin and lanolin derivatives, and fatty alcohol ethoxylates, emollient oils, fatty acids, fatty alcohols, and their esters. Other examples of suitable bases include glycerin, sortibal aloe, poylglycols, polyethylene glycol, polyoxyethylene and polyethylene oxide.

In some embodiments, the composition includes a cationic or ionic surfactant with a hydrophile-lipophile balance (“HLB”) of from about 12 to about 18. In some embodiments, the composition includes a cationic or ionic surfactant with an HLB of from about 13 to about 16.

### Aqueous Agent

In some embodiment, the disclosed compositions may include an aqueous agent. The aqueous agent can be any suitable material that can dissolve the active ingredients and co-ingredients and hydrate the biocidal system into the absorbent carrier and ultimately deliver the composition to the source of the odor. Water is a convenient aqueous agent for liquid embodiments of the disclosed composition.

### Formulations

The following are non-limiting examples of the disclosed compositions:

TABLE 1

| Ingredients | Amount (wt %) |
|---|---|
| Malic Acid | 0.10-0.75 |
| pH buffer | 0.05-1.00 |
| Thickener | 0.25-4.00 |
| Color | trace |
| Aqueous agent | balance |

In some embodiments, the thickener present in the composition set forth in Table 1 may be replaced with a foaming agent (˜1.25 wt %).

TABLE 2

| Ingredients | Amount (wt %) |
|---|---|
| Cetyl pyridinium | 0.10-1.00 |
| chloride | |
| pH buffer | 0.05-1.00 |
| PEG 6 | 0.35-5.00 |
| Thickener | 0.25-4.00 |
| color | Trace amounts |
| Aqueous agent | Remaining balance |
| | of composition |

### Methods of Hydrating an Absorbent Carrier

An embodiment of the invention is directed to a method of infusing or hydrating an absorbent carrier with an odor eradicating composition. The method includes applying the composition onto the absorbent carrier. The composition may be applied by, for example, a hose or nozzle. In a typical embodiment. the absorbent carrier is fully immersed in the composition. After the absorbent carrier has been fully immersed in the composition, the absorbent carrier is dried. Drying may be accomplished in various ways, including, for example, spray drying the composition onto the absorbent carrier.

### Methods of Use

Absorbent carriers that have been treated with the compositions disclosed herein can be used in variety of applications. For example, the treated absorbent carriers may be incorporated into cat-litter box. In some embodiments, the cat litter may consist of the treated absorbent carrier. In some embodiments, the treated absorbent carrier may be mixed into an existing cat litter.

In another embodiment, the treated absorbent carrier may be incorporated into a poultry pen. In some embodiments, the absorbent carrier may include sawdust that has been treated with one or more of the compositions disclosed herein. The treated sawdust may be distributed across a floor of the poultry pen.

In another embodiment, the treated absorbent carrier may be incorporated into a synthetic turf. In some embodiments, the absorbent carrier may include silica that is mixed into the synthetic turf.

In another embodiment, an air filter for a heating, ventilation, and air conditioning system may include a treated absorbent carrier. For example, the absorbent carrier may include charcoal.

In another embodiment, a kitchen air cleanser may include a treated absorbent carrier. In some embodiments, the absorbent carrier may be a polymer.

The compositions of the present invention can be incorporated in an article of manufacture that includes the composition plus a dispenser (e.g., a spray or pump dispenser). The dispenser can be any of the manually activated devices for producing a spray of liquid droplets as known in the art, such as, for example, trigger-type, pump-type, non-aerosol self-pressurized, and aerosol-type spray devices, as well as electrodynamic spray devices. It is preferred that at least 80%, and more preferably at least 90%, of the liquid droplets have a particle size of larger than about 30 μm.

The odor-control compositions herein can be used by distributing, e.g., by placing the aqueous solution into a dispensing means, for example a spray dispenser, and dispensing an effective amount onto the desired surface or article. An effective amount as defined herein means an amount sufficient to absorb odor to the point that it is not discernible by the human sense of smell yet not so much as to saturate or create a pool of liquid on said article or surface and so that when dry there is no visual deposit readily discernible. Distribution can be achieved by using a spray device, a roller, a pad, etc.

Household surfaces that can be treated herewith include countertops, cabinets, walls, floors, bathroom surfaces and kitchen surfaces. Household appliances that be treated herewith include refrigerators, freezers, washing machines, automatic dryers, ovens, microwave ovens, dishwashers, garbage cans, waste disposal units etc. Fabric and/or fabric articles that can be treated herewith include clothes, shoes, curtains, drapes, shower curtains, upholstered furniture, carpeting, bed linens, bath linens, tablecloths, sleeping bags, tents, car interior, e.g., car carpet, fabric car seats, etc. Other items that can be treated herewith include cat litter, pet bedding, pet houses and household pets themselves.

### Working Examples

The following procedures were used to evaluate the disclosed compositions against various microorganisms. The composition tested in each of the below examples included 0.25 wt % malic acid, 0.10 wt % pH buffer, and 0.5 wt % thickener. The results below further indicate the effectiveness of the disclosed compositions.

Bacterial testing was completed at Biological Consulting Services of North Florida, Inc. on E. coli, and E. faecalis. The results are listed in Table 3.

TABLE 3

| Sample | Control units (cfu/ml) | Results units (cfu/ml) |
|---|---|---|
| E. coli | 9.3 × 10⁵ | <0.5 |
| Enterococcus faecalis | 1.1 × 10⁶ | <0.5 |

A Draegar Pump and ammonia detection Draegar tubes were used to determine gas levels. The Draegar pump is a standard bellow style gas sampling pump. It drags a calibrated 100 ml sample of the gas through short-term Draegar tubes. Table 4 shows the results of ammonia expressed as parts per million (ppm). Urine samples were measured for ammonia gas before and after application was done on cat litter. The study times were less than 1 hour after application and 5 days after application. The table below shows those results:

TABLE 4

| | | |
|---|---|---|
| Urine | 1 Hour | 5 Days |

| Samples | Before | After | Before | After |
|---|---|---|---|---|
| Sample 1 | 15 ppm | 2 ppm | 170 ppm | 4 ppm |
| Sample 2 | 18 ppm | 2 ppm | 210 ppm | 5 ppm |
| Sample 3 | 15 ppm | 2 ppm | 200 ppm | 4 ppm |
| Sample 4 | 20 ppm | 2 ppm | 190 ppm | 4 ppm |
| Sample 5 | 20 ppm | 2 ppm | 210 ppm | 5 ppm |

Thus, it can be seen that compositions of the claimed invention are capable of reducing the presence of ammonia gas by about 90% to 98% depending upon the time of treatment, i.e., less than one hour or five days. Treatment times of as little as ten (10) minutes can have results at or near the results of those measured at one (1) hour.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are with the scope of this disclosure.

What is claimed is:

1. A method of treating odors comprising:
applying an odor eliminating composition to an absorbent carrier;
wherein the odor eliminating composition comprises:
a biocidal system;
a pH buffer.

2. The method of claim 1, further comprising dehydrating the absorbent carrier prior to the applying.

3. The method of claim 1, wherein the biocidal system comprises from about 0.01% to about 20% by weight of the odor eliminating composition.

4. The method of claim 3, wherein a primary biocide comprises from about 0.01% to about 25% by weight of the biocidal system.

5. The method of claim 4, wherein the primary biocide comprises one or more of a quaternary ammonium compound, copper, zinc, silver, chloride salts, perchlorates, hypochlorites, sulfates, sulfites, bisulfates, and bisulfites.

6. The method of claim 4, wherein the primary biocide comprises an organic acid.

7. The method of claim 1, wherein the pH buffer is a biocidal, non-corrosive, acid composition comprising a proton count range of from 1.0×10²⁰ to 9.9×10²⁶ and an embodied conductivity range of from 250 mV to 1500 mV and a 1% solution of the composition has a pH under 2.0.

8. The method of claim 1, wherein the pH buffer comprises between about 0.01% to about 25% by weight of the odor eliminating composition.

9. The method of claim 1, wherein the absorbent carrier comprises a polymer crystal with a plurality of pores, wherein diameters of each of the plurality of pores is greater than 10 Angstroms.

10. The method of claim 1, wherein the absorbent carrier comprises about 0.001% to about 90% by weight of the odor eliminating composition.

11. The method of claim 1, wherein the absorbent carrier is selected from cotton, paper, charcoal, clay, soil, manure, walnut shells, wheat and wheat by products, saw dust; cellulosic materials, polymers, hydrogels, upsalite, silica; carbohydrates, lipids, proteins, polymers, acrylates, polyvinyl alcohol, silicas, silicates, calcium chloride, and rock salt.

12. The method of claim 11, wherein the absorbent carrier is incorporated with a binder or a porous matrix material.

13. A composition for eliminating odors, the composition comprising:
an absorbent carrier;
a biocidal system, wherein the biocidal system comprises from about 0.01% to about 20.0% by weight of the odor eliminating composition; and
a pH buffer.

14. The composition of claim 13, wherein the absorbent carrier is selected from cotton, paper, charcoal, clay, soil, manure, walnut shells, wheat and wheat by products, saw dust;
cellulosic materials, polymers, hydrogels, upsalite, silica; carbohydrates, lipids, proteins, polymers, acrylates, polyvinyl alcohol, silicas, silicates, calcium chloride, and rock salt;

15. The composition of claim 13, wherein a primary biocide comprises from about 0.01% to about 20% by weight of the biocidal system.

16. The composition of claim 13, wherein a primary biocide comprises from about 0.01% to about 25% by weight of the biocidal system.

17. The composition of claim 16, wherein the primary biocide comprises one or more of a quaternary ammonium compound, copper, zinc, silver, chloride salts, perchlorates, hypochlorites, sulfates, sulfites, bisulfates, and bisulfites.

18. The composition of claim 13, wherein the pH buffer is a biocidal, non-corrosive, acid composition comprising a proton count range of from 1.0×10²⁰ to 9.9×10²⁶ and an embodied conductivity range of from 250 mV to 1500 mV and a 1% solution of the composition has a pH under 2.0.

19. The composition of claim 13, wherein the pH buffer comprises between about 0.01% to about 25% by weight of the odor eliminating composition.

20. The composition of claim 13, wherein the absorbent carrier comprises about 0.001% to about 90% by weight of the odor eliminating composition.
